(19) United States
(12) Patent Application Publication
(10) Pub. No.: US 2026/0345129 A1
(43) Pub. Date: Nov. 5, 2026

(54) SYSTEMS AND METHODS FOR DIAGNOSING RECTAL SUPPORT DEFECTS

(71) Applicant: Altyx Medical, Inc., Evanston, IL (US)

(72) Inventors: Ghazaleh Rostami NIA, Evanston, IL (US); Roger GOLDBERG, Evanston, IL (US); Douglas SCHERR, Evanston, IL (US)

(21) Appl. No.: 19/115,514

(22) PCT Filed: Sep. 28, 2023
(86) PCT No.: PCT/US2023/075341

§ 371 Date: Mar. 26, 2025

Related U.S. Application Data
(60) Provisional application No. 63/410,999, filed on Sep. 28, 2022.

(51) Int. Cl. A61B 8/00; A61B 8/08; G16H 50/20

(52) U.S. Cl. CPC A61B 8/5223; A61B 8/085; G16H 50/20

(57) ABSTRACT

Systems and methods for improving the efficiency and effectiveness of dynamic ultrasound in the diagnosis and evaluation of ODS patients are described herein. For example, the present technology includes using artificial intelligence architectures and/or machine learning models to provide real time feedback to a user regarding ultrasound probe positioning to better capture patient pelvic floor anatomy. The present technology also includes using artificial intelligence architectures and/or machine learning models to analyze ultrasound video or images of a patient to objectively evaluate to the patient's pelvic floor anatomy.

### CROSS-REFERENCE TO RELATED APPLICATION(S)

The present application is a National Stage of International Patent Application No. PCT/US23/75341, filed Sep. 28, 2023, which claims priority to U.S. Provisional Patent Application No. 63/410,999, filed Sep. 28, 2022, the disclosures of which are incorporated by reference herein in their entireties.

### TECHNICAL FIELD

The present technology is generally directed to systems, devices, and methods for diagnosing and/or treating rectal support defects.

### BACKGROUND

Obstructed defecation (OD) and obstructed defecation symptoms (ODS) are commonly encountered in urogynecology settings, with around 7% of the adult female population reporting ODS. Patients with ODS that have observable structural defects are often treated through a variety of surgical techniques that attempt to repair the pelvic floor and provide support to the rectum. However, despite the widespread nature of ODS, there is a lack of reliable and accessible tests for identifying structural causes contributing to ODS, thereby making it difficult to identify suitable candidates for surgical intervention. This results in some patients who may benefit from surgical intervention failing to receive adequate care, while others who would not benefit from surgical intervention receiving invasive and costly surgical treatment that provides little to no relief from ODS. Accordingly, a need exists for improved systems and methods for diagnosing rectal support defects associated with ODS and identifying candidates for surgical intervention.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIGS. 1A and 1B illustrate representative pelvic anatomy of a normal patient.

FIGS. 2A and 2B illustrate representative pelvic anatomy of a patient with pelvic floor defects associated with obstructed defecation symptoms.

FIG. 3 is a schematic illustration of patient treatment system including a patient imaging system and a remote computing system configured in accordance with select embodiments of the present technology.

FIG. 4 is a flow diagram of a method of providing medical care to a patient in accordance with embodiments of the present technology.

FIGS. 5A-5C are representative ultrasound frames showing target anatomical landmarks and target points on the target anatomical landmarks in accordance with select embodiments of the present technology.

### DETAILED DESCRIPTION

Dynamic ultrasound has recently been introduced as a useful tool for diagnosing and evaluating ODS patients. In particular, dynamic ultrasound can be used to assess pelvic floor anatomy, including assessing, among other things, rectocele, enterocele, rectal intussusception, and rectal prolapse. Despite the advantages associated with dynamic ultrasound, adoption of this technology as a tool for diagnosing and evaluating ODS patients has been hindered by the complexity of the procedure required to obtain useful diagnostic information, the labor involved in segmenting and analyzing the ultrasound data, and the inter- and intra-user variability associated with such procedures. For example, many urogynecologists lack the knowledge and training to perform dynamic ultrasound procedures. And even for urogynecologists that have sufficient knowledge and training, the process of obtaining and analyzing dynamic ultrasound is time consuming and provides little objectivity.

The present technology is expected to address the foregoing problems to improve the efficiency and effectiveness of using dynamic ultrasound to diagnose and evaluate ODS patients. For example, in some embodiments the present technology uses artificial intelligence architectures and/or machine learning models to provide real time feedback to a user regarding probe positioning. For example, the present technology can automatically analyze, in real time, ultrasound video or images and, based on the video/images, instruct the user to change a position or setting of the probe to obtain better video or images of patient pelvic floor anatomy. In some embodiments, the present technology also uses artificial intelligence architectures and/or machine learning models to analyze ultrasound video or images of a patient to objectively evaluate the patient's pelvic floor anatomy. For example, the present technology can automatically identify target anatomical landmarks, measure distances associated with the target anatomical landmarks, and calculate objective metrics for evaluating the patient's pelvic floor anatomy. As described in detail throughout this Detailed Description, the present technology includes additional systems and methods for improving the efficiency and effectiveness of dynamic ultrasound for diagnosing and evaluating ODS patients.

Further aspects and advantages of the devices, methods, and uses will become apparent from the ensuing description that is given by way of example only.

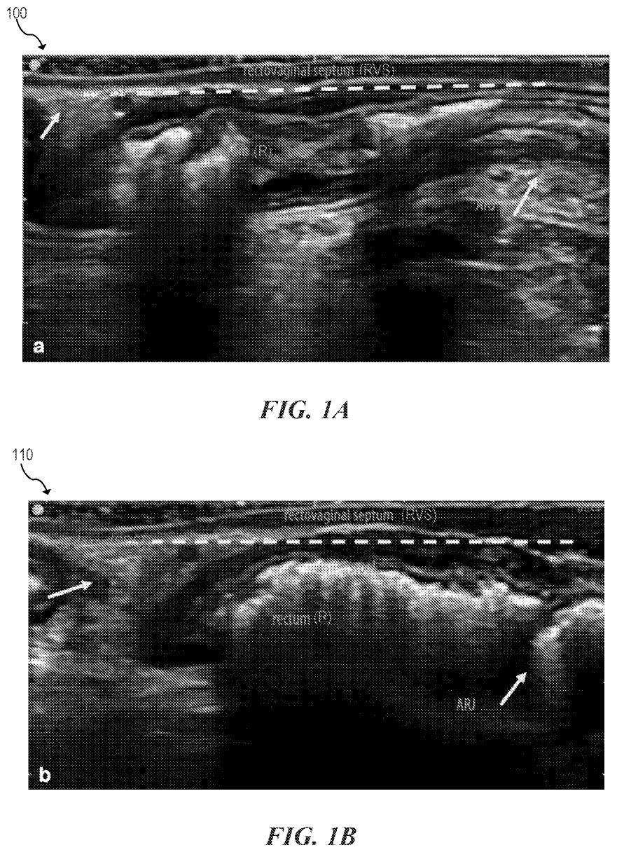

The terminology used in the description presented herein is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and/or claims but that are not described in detail with respect to FIGS. 1A-5C.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. The term “substantially” or grammatical variations thereof refers to at least about 50%, for example, 75%, 85%, 95%, or 98%.

The headings provided herein are for convenience only and do not interpret the scope or meaning of the claimed present technology.

### A. Obstructive Defecation Symptoms and Rectal Hypermobility

The process of normal rectal continence and evacuation are regulated through a coordinated interaction of neuromuscular pathways that, during defecation, cause intraabdominal pressure to increase and push feces toward and through the anal sphincter. Various connective structures provide mechanical support to the rectum to assist this process. For example, the rectum is supported apically by the anterior peritoneal reflection, is supported laterally by lateral ligaments or stalks of the rectum, is supported anteriorly by the visceral pelvic fascia of Denonvilliers, and is supported posteriorly by the rectosacral fascia. These connective structures help restrain the rectum and prevent excessive rectal mobility during straining (e.g., Valsalva straining efforts). In contrast, some patients with ODS have structural anatomical defects that may impair the ability of the patient to properly defecate. For example, some patients with ODS present with loss of, or malfunctioning of, connective tissues supporting the rectum. Patients with impaired structural support may exhibit rectal hypermobility and/or rectovaginal septum folding during Valsalva straining efforts.

FIGS. 1A and 1B are images showing representative healthy patient anatomy at rest and during Valsalva, respectively. More specifically, FIG. 1A is a first ultrasound image 100 showing select anatomical landmarks of a pelvic region in a representative healthy patient at rest, and FIG. 1B is a second ultrasound image 110 showing select anatomical landmarks of the pelvic region in the representative healthy patient anatomy during Valsalva.

Referring collectively to FIGS. 1A and 1B, the image 100 and the image 110 include certain anatomical landmarks, including a rectum R, a rectovaginal septum RVS, a posterior cul de sac CDS, and an anorectal junction ARJ. The cul de sac CDS and the anorectal junction ARJ are positioned at opposite ends of the infra-peritoneal section of the rectum R (referred to as the “infra-peritoneal rectum R”) and the rectovaginal septum RVS. Thus, a distance between the cul de sac CDS and the anorectal junction ARJ can be used to approximate a length of the infra-peritoneal rectum R and/or the rectovaginal septum RVS. As described in greater detail below, other anatomical landmarks, such as the levator plate (not shown in FIGS. 1A and 1B), can also be used to approximate a length of the infra-peritoneal rectum R and/or rectovaginal septum RVS. For example, in some embodiments a distance between a center of the cul de sac CDS and a center of the levator plate can be used to approximate a length of the infra-peritoneal rectum R and/or the rectovaginal septum RVS (e.g., because the levator plate is at the same or at least similar anatomical level as the anorectal junction ARJ).

As seen by comparing FIGS. 1A and 1B, the rectum R and the rectovaginal septum RVS maintain a relatively consistent length during rest (FIG. 1A) and during Valsalva (FIG. 1B). This is because the various connective tissues described previously restrain the rectum R and the rectovaginal septum RVS from sliding/compressing distally during Valsalva. This reduces and/or prevents excessive changes in the length of the infra-peritoneal rectum R and the rectovaginal septum RVS between rest and Valsalva. Moreover, because the cul de sac CDS and the anorectal junction ARJ are positioned at opposite ends of the infra-peritoneal rectum R and the rectovaginal septum RVS, a distance between the center of the cul de sac CDS and the center of the anorectal junction ARJ decreases only slightly during Valsalva in patients with healthy/normal pelvic floor anatomy.

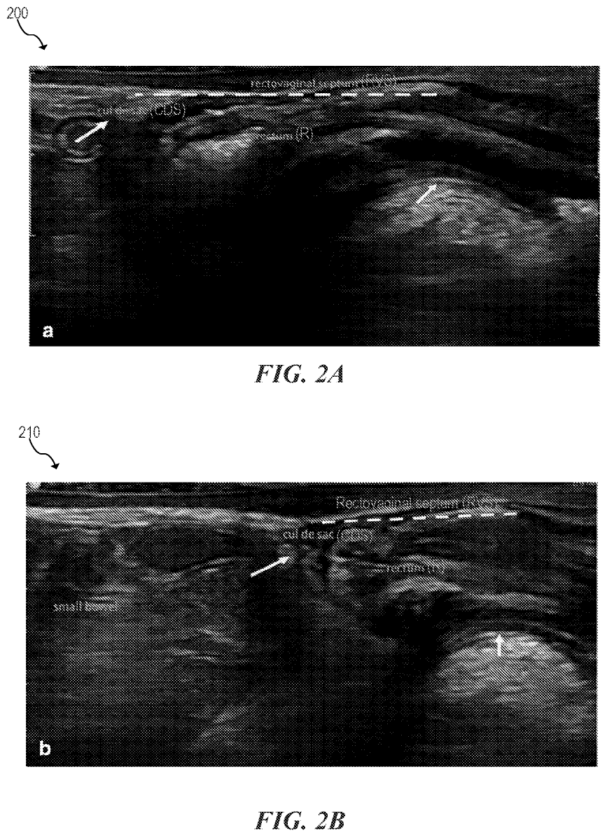

FIGS. 2A and 2B are images showing representative patient anatomy for a patient with ODS at rest and during Valsalva, respectively. More specifically, FIG. 2A is a first ultrasound image 200 showing select anatomical landmarks of a pelvic region of an ODS patient at rest, and FIG. 2B is a second ultrasound image 210 showing select anatomical landmarks of the pelvic region of the ODS patient during Valsalva.

As seen by comparing FIGS. 2A and 2B, both the rectum R and the rectovaginal septum RVS are compressed and slide distally (e.g., toward the anorectal junction ARJ) during Valsalva. As also shown in FIGS. 2A and 2B, the posterior cul de sac CDS descends toward the anorectal junction ARJ during Valsalva. Without being bound by theory, one potential mechanism underlying the sliding of the posterior cul de sac CDS and the compression of the rectum R and rectovaginal septum RVS in ODS patients may involve defects in lateral and/or anterior support structures, or other rectal support structures.

The inventors of the present application have created an algorithm to quantify the degree of rectal sliding/hypermobility observed during Valsalva. The algorithm quantifies a compression ratio using the following equation:

$$Compression Ratio = 100 \times \frac{( RVA length at rest - RVA length at Valsalva )}{RVS Length at Rest} \qquad ( 1 )$$

Because the length of the rectovaginal septum and infra-peritoneal section of the rectum are correlated, the foregoing equation can also use infra-peritoneal rectal length (IPRL) in place of rectovaginal septum length.

$$Compression Ratio = 100 \times \frac{( IPRL at rest - IPRL at Valsalva )}{IPRL at Rest} \qquad ( 2 )$$

Moreover, because the RVS length and infra-peritoneal rectum length can be approximated using a distance between a center of the posterior cul de sac CDS and the anorectal junction AJ, the compression ratio can also be calculated using the following equation:

$$Compression Ratio = 100 \times \frac{( CDS to ARJ length at rest - CDS to ARJ length at Valsalva )}{CDS to ARJ length at Rest} \qquad ( 3 )$$

A high compression ratio indicates greater RVS folding, and thus greater rectal mobility during Valsalva. This in turn indicates likely structural support defects in the rectum. That is, ODS patients with a high compression ratio can be identified as having likely structural defects that contribute to their ODS. For example, the present inventors identified in a previous study that the risk of ODS was 32 times greater in patients with a compression ratio of greater than or equal to (≥) 14 than in patients with a compression ratio of less than (<) 14. Such patients presenting with a high compression ratio may thus be identified as candidates for surgical intervention to provide rectal support.

### B. Representative Systems and Methods for Diagnosing Rectal Support Defects

Despite the use of dynamic ultrasound and introduction of the compression ratio as techniques for quantifying ODS structural defects and identifying surgical candidates, many physicians that treat ODS patients lack proper training to perform dynamic ultrasounds and/or calculate compression ratios from ultrasound images. As a result, the benefits of using dynamic ultrasound and calculated compression ratios to evaluate and quantify ODS structural defects and identifying surgical candidates have not been fully met. As set forth below, the present technology includes systems and methods that utilize artificial intelligence to (1) provide intra-ultrasound feedback to ensure the ultrasound technician is obtaining adequate imaging of patient pelvic floor anatomy, and (2) automatically analyze and calculate a compression ratio based on the obtained imaging. Without being bound by theory, this is expected to increase access to dynamic ultrasound as a viable testing procedure for quantifying ODS structural defects and developing successful treatment plans for affected patients.

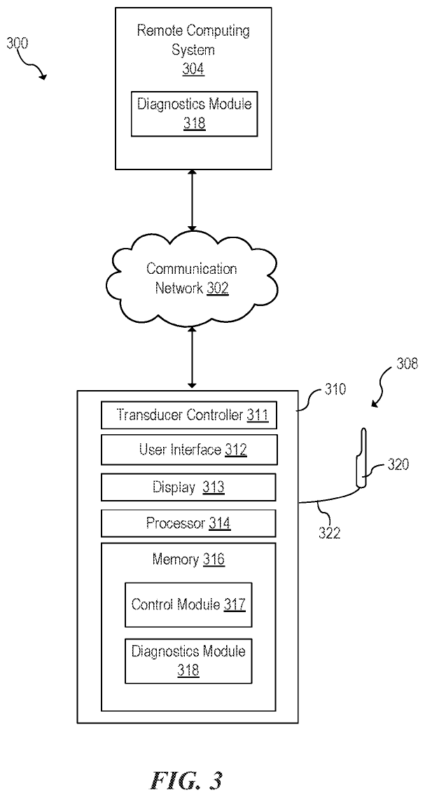

FIG. 3 is a schematic illustration of a representative patient imaging or treatment system 300 (“the system 300”) configured in accordance with select embodiments of the present technology. As shown, the system 300 includes a remote computing system 304, a communication network 302, and an ultrasound system 308. The remote computing system 304 can be implemented as a computing device, server, and/or a distributed cloud-based storage system, and can store various software modules having instructions for executing the methods and techniques described herein. The ultrasound system 308 can be a conventional ultrasound system for obtaining patient images, and/or a modified ultrasound system having various software modules for executing some of the methods described herein. The communication network 302 can establish a link between the remote computing system 304 and the ultrasound system 308 to provide data transmission therebetween. As described below, the system 300 can be used to examine patients to determine whether the patients are suitable candidates for ODS surgery.

The ultrasound system 308 can include a transducer probe 320 (“the probe 320”) for transmitting and receiving ultrasound waves, and a housing 310 containing various hardware and software components for controlling the probe 320, processing data received from the probe 320, displaying images obtained via the probe 320, or the like. The transducer probe 320 can be an intravaginal probe, an intrarectal probe, or another suitable transducer for obtaining ultrasound images or videos of a pelvic region of the patient (not shown). For example, the probe 320 can include a 3D 20R3 Anorectal Transducer from BK Medical (Burlington, MA), or any other suitable transducer probe from BK Medical or another manufacturer. In operation, the probe 320 can be inserted into patient's vagina or rectum to obtain ultrasound images and/or videos of the patient's pelvic region.

The housing 310 can include a transducer controller 311, a user interface 312, a display 313, a processor 314, and a memory 316. The controller 311 can control signals (e.g., electrical signals) sent to and received from the probe 320. The user interface 312 enables a user (e.g., ultrasound technician) to control various settings for the ultrasound system 308. The display 313 can include a screen for displaying one or more images or videos produced by the ultrasound system 308. The memory 316 can store one or more computer-executable instructions (e.g., software modules) executable by the processor 314 to control various operations of the ultrasound system 308, including controlling the ultrasound system 308 to perform the methods described herein. For example, the memory 316 can store a control module 317 for controlling various operations of the ultrasound system 308, such as processing data received from the probe 320, generating an image or video using the data received from the probe 320, displaying the generated image or video via the display 313, or other standard operations performed by ultrasound systems. Although described as being contained within a housing 310, in some embodiments one or more of the foregoing components are contained within distinct housings.

In some embodiments, the ultrasound system 308 is configured to provide dynamic ultrasound. In such embodiments, the patient can be instructed to perform a specific action or movement when the probe 320 is located at a desired position and activated. In the context of the present technology, this may include instructing the patient to perform a Valsalva straining maneuver with the probe 320 inserted into the patient's vagina or rectum. In this way, the ultrasound system 308 can capture video or images at various patient states, including at rest and during Valsalva.

The system 300 also includes a diagnostics module 318. The diagnostics module 318 can include software, algorithms, machine-learning models, artificial intelligence architectures, or the like for performing select operations, as described in detail below. In some embodiments, the diagnostics module 318 is stored at the remote computing system 304. In other embodiments, the diagnostics module 318 is stored within the memory 316 of the ultrasound system 308. In some embodiments, a first portion (e.g., first computer executable instructions) of the diagnostics module 318 can be stored at the remote computing system 304, and a second portion (e.g., second computer executable instructions) of the diagnostics module 318 can be stored within the memory 316 of the ultrasound system 308. Accordingly, for purposes of illustration, the diagnostics module 318 is shown in FIG. 3 at both the ultrasound system 308 and the remote computing system 304—one skilled in the art, however, will appreciate that the diagnostics module 318 can be stored solely at the remote computing system 304, solely at the ultrasound system 308, or at both the remote computing system 304 and the ultrasound system 318. In yet other embodiments, the diagnostics module 318 can be stored at other locations, such as at or on another networked device, server, or system. Regardless of where the diagnostics module 318 is stored, and as described in greater detail below, the diagnostics module 318 can be configured to (a) generate feedback in real time to assist an ultrasound technician in positioning the probe 320 to obtain desired images or videos of the patient, and/or (b) automatically analyze and calculate a compression ratio based on the obtained images or videos of the patient.

The diagnostics module 318 can be configured to provide feedback to an ultrasound technician or other user during an imaging session. The feedback can be related to the positioning of the probe 320 relative to the patient, e.g., to ensure the ultrasound images contain target anatomical landmarks (e.g., the rectum, the rectovaginal septum, the cul de sac, the anorectal junction, the levator plate, etc.). For example, the diagnostics module 318 can (a) analyze images or video obtained by the user, (b) detect the presence or absence of target anatomical landmarks, and (c) based on (b), instruct the user to advance the probe 320 deeper into the patient, retract the probe 320 from the patient, rotate the probe 320, adjust an angle of the probe 320, adjust a setting of the signals emitted from the probe 320, or the like. The feedback can be provided to the user in real-time or substantially real-time (e.g., less than 5 second delay, less than 2 second delay, less than 1 second delay, less than 0.5 second delay, etc.).

The diagnostics module 318 can also be configured to analyze recorded ultrasound video or images. For example, the diagnostics module 318 can (a) automatically convert ultrasound video into individual frames or images, (b) analyze the individual frames or images to identify target anatomical landmarks, (c) segment the target anatomical landmarks within the individual frames, (d) identify target points (e.g., center points, edges, etc.) of the target anatomical landmarks, (e) extract measurements associated with the target points and/or target anatomical landmarks from the individual frames, and/or (f) compute pelvic floor metrics (e.g., a compression ratio) based on the extracted measurements. In some embodiments, the diagnostics module 318 is configured to automatically perform one or more of the foregoing operations in real time or substantially real-time.

In some embodiments, the diagnostics module 318 includes one or more trained artificial intelligence (AI) architectures or machine learning (ML) models for performing various operations described herein. The AI architectures and/or ML models can include neural networks, convolutional neural networks, deep learning algorithms, or the like. In some embodiments, the AI architecture includes a U-net architecture. Suitable AI techniques that can be incorporated into the AI architectures and/or ML models include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems. In some embodiments, for example, the diagnostics module 318 includes a trained AI architecture or ML model that can automatically analyze ultrasound images or videos and provide feedback to identify anatomical landmarks and, based on the analysis, provide instructions to an ultrasound technician to adjust a positioning of the transducer probe 320 in order to capture target anatomical landmarks. Similarly, the diagnostics module 318 can include a trained AI architecture or ML model that (a) converts ultrasound video into individual frames or images, (b) analyzes the individual frames or images to identify target anatomical landmarks, (c) segments the target anatomical landmarks within the individual frames, (d) identifies target points (e.g., center points, edges, etc.) of the target anatomical landmarks, (e) extracts measurements associated with the target points and/or target anatomical landmarks from the individual frames, and/or (f) computes pelvic floor metrics (e.g., a compression ratio) based on the extracted measurements.

Some embodiments of an ML model include a construct that is trained using training data to make predictions or provide probabilities for new data items, whether or not the new data items were included in the training data. For example, training data for supervised learning can include items with various parameters and an assigned classification. A new data item can have parameters that a model can use to assign a classification to the new data item. As another example, a model can be a probability distribution resulting from the analysis of training data, such as a likelihood of an n-gram occurring in a given language based on an analysis of a large corpus from that language.

In some embodiments, an ML model can be a neural network with multiple input nodes that receive input data, such as an ultrasound image. The input nodes can correspond to functions that receive the input and produce results. These results can be provided to one or more levels of intermediate nodes that each produce further results based on a combination of lower-level node results. A weighting factor can be applied to the output of each node before the result is passed to the next layer node. At a final layer, (“the output layer”) one or more nodes can produce a value classifying the input that, once the model is trained, label object locations and classifications within an ultrasound image. Some embodiments of such neural networks, known as deep neural networks, can have multiple layers of intermediate nodes with different configurations, can be a combination of models that receive different parts of the input and/or input from other parts of the deep neural network, or are convolutions-partially using output from previous iterations of applying the model as further input to produce results for the current input.

An ML model can be trained with supervised learning, where the training data includes, for example, an ultrasound image labeled with target anatomical landmarks. A representation of the ultrasound image can be provided to the model. Output from the model can be compared to the desired output for that image, for example by determining whether the model correctly detected the location and classification of each target anatomical landmark. Based on the comparison, the model can be modified, such as by changing weights between nodes of the neural network or parameters of the functions used at each node in the neural network (e.g., applying a loss function). After applying each of the labeled ultrasound images in the training data and modifying the model in this manner, the model can be trained to evaluate new ultrasound image data.

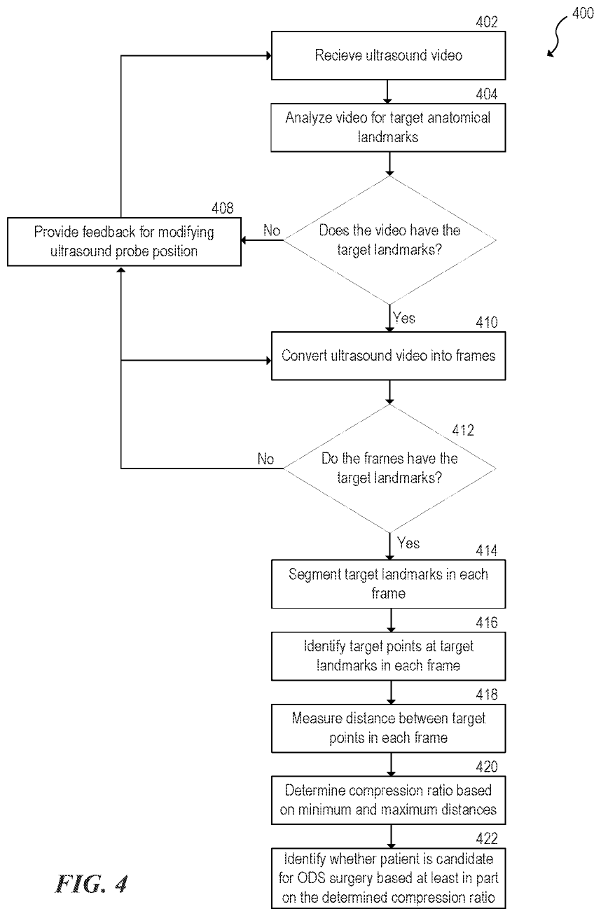

FIG. 4 is a flow diagram illustrating a method 400 for providing medical care to patient in accordance with some embodiments of the present technology. As one skilled in the art will appreciate from the following, the method 400 can be performed by one or more computing systems executing computer-executable instructions stored on a non-transitory computer-readable medium. For example, in some embodiments some or all of the method 400 can be performed by the system 300 of FIG. 3 executing computer-readable instructions stored on or within the diagnostics module 318.

The method 400 can begin at block 402 by receiving ultrasound video of a patient. In some embodiments, receiving the ultrasound video of the patient includes recording the ultrasound video of the patient. For example, a transducer probe connected to an ultrasound machine may be inserted into the patient's vagina or rectum to obtain a video of the patient's pelvic anatomy. In some embodiments, the ultrasound video is received over a communication network (e.g., the communication network 302 of FIG. 3).

The method 400 can continue at block 404 by analyzing the ultrasound video to identify one or more target anatomical landmarks. In some embodiments, the one or more target anatomical landmarks are associated with a pelvic region of the patient. For example, in some embodiments the target anatomical landmarks include one or more of the rectum, the rectovaginal septum, the cul de sac, the levator plate, and/or the anorectal junction. In other embodiments, other suitable anatomical landmarks can be utilized. The target anatomical landmarks can be preselected. For example, in some embodiments, the target anatomical landmarks can be user-specified (e.g., a user inputs the target anatomical landmarks before the operation in block 404 is performed). In such embodiments, the operation at block 404 includes analyzing the ultrasound video to confirm the preselected target anatomical landmarks are included within the video. In some embodiments, analyzing the ultrasound video to identify the target anatomical landmarks at block 404 includes using one or more trained AI architectures and/or ML models to identify the target anatomical landmarks. Further, in some embodiments additional steps may be utilized to supplement standard object detection models. These steps can include, for example, certain training data that is needed, certain ways the training data or production data needs to be preprocessed for input to the model, and/or any refinement steps that are needed to apply a standard object detection model in this particular application (e.g., to improve handling of ultrasound image data, to account for anatomical differences from patient to patient, to handle slight differences in how an ultrasound probe is inserted, etc.). In such embodiments, the AI architecture and/or ML model can be the same or different than the AI architecture and/or ML model used to perform other operations of the method 400.

If the ultrasound video does not include the target anatomical landmarks, the method 400 can proceed at block 408 by providing feedback for modifying a position of the ultrasound probe. The feedback can include specific instructions for moving the probe so that the probe will capture a video with the target anatomical landmarks. For example, the feedback may include instructions to advance the probe deeper into the patient by a specific distance, retract the probe from the patient by a specific distance, rotate the probe a specific number of degrees, adjust an angle of the probe, adjust a setting of the signals emitted from the probe, or the like. The feedback can be displayed on a screen of the ultrasound system (e.g., the display 313 of the ultrasound system 308 of FIG. 3) or another suitable display (e.g., mobile phone, tablet, computer, etc.). In some embodiments, providing feedback at block 408 includes using one or more trained AI architectures and/or ML models to provide the feedback. By way of example, in one embodiment the feedback process can include using an ML model, which is trained to detect landmarks other than the target landmark in an image/video, to identify any additional landmarks it can see within the video/a given frame. If a landmark other than the target landmarks is identified, this landmark is compared to a basic anatomical map. For example, it is expected that certain landmark(s) will be at a certain position in the frame for a target landmark to be visible. The system thus compares the current position of the identified landmark to its expected position and generates a mapping from the current position to expected position that translates into a specific instruction for moving the probe. Further, if no landmarks are identified in the frame, a default instruction may be output by the system, e.g., instructing a user to fully remove the probe and reinsert, which enables the system to identify landmarks as the probe is being reinserted. In some embodiments, the trained AI architecture and/or trained ML model can be the same or different than the AI architecture and/or ML model used to perform other operations of the method 400.

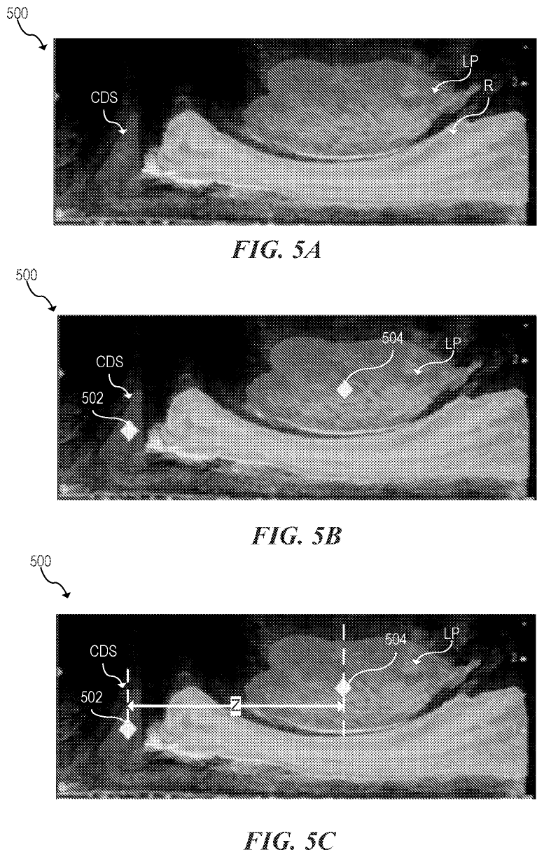

If the ultrasound video includes the target anatomical landmarks, the method 400 can proceed at block 410 by converting the ultrasound video into individual frames. The frames can be generated at intervals of from about 1 frame/second to about 10,000 frames/second, or other suitable intervals. In some embodiments, converting the ultrasound video into individual frames includes using one or more trained AI architectures and/or ML models to convert the ultrasound video into individual frames. In such embodiments, the AI architecture and/or ML model can be the same or different than the AI architecture and/or ML model used to perform other operations of the method 400. An example of an ultrasound frame converted from an ultrasound video in accordance with the present technology is provided below in FIGS. 5A-5C.

Once the ultrasound video is converted into individual frames, the method 400 can continue at block 412 by determining whether the frames include each of the target anatomical landmarks. The target anatomical landmarks can be the same as the target anatomical landmarks from the operation at block 404. For example, the target anatomical landmarks can include the rectum, the rectovaginal septum, the cul de sac, the levator plate, and/or the anorectal junction. If each frame includes all of the target anatomical landmarks, the method 400 can proceed to the operation at block 414, described below. If each frame does not include all of the target anatomical landmarks, the method 400 can return to the operation at block 410, and the operations at blocks 410 and 412 can be iteratively repeated until each frame includes all of the target anatomical landmarks. In other embodiments, if each frame does not include all of the target anatomical landmarks, the method 400 can return to the operation at block 408 to provide user feedback for probe positioning, and the operations at blocks 402-412 can be iteratively repeated until each frame includes all the target anatomical landmarks.

Although the operation at block 412 is described as determining whether each frame includes each target anatomical landmark, in some embodiments a threshold value is used. For example, the operation at block 412 may include determining whether at least 80%, at least 85%, at least 90%, at least 95%, at least 98%, or at least 99% of the frames have all of the target anatomical landmarks. In such embodiments, the method 400 can proceed to the operation at block 414 if the target threshold is reached (e.g., if at least 80% of the frames include each target anatomical landmark, if at least 85% of the frames include each target anatomical landmark, etc.). In some embodiments, determining whether the frames include the target anatomical landmarks at block 412 includes using a trained AI architecture and/or ML model to analyze the frames and identify the target anatomical landmarks. In such embodiments, the AI architecture and/or ML model can be the same or different than the AI architecture and/or ML model used to perform other operations of the method 400.

The method 400 can continue at block 414 by segmenting the target anatomical structures in each of the frames. In embodiments in which a threshold value is used at block 412, the frames that do not include each of the target anatomical landmarks can be excluded from segmentation. In some embodiments, segmenting the target anatomical structures at block 414 includes using a trained AI architecture and/or ML model to segment the target anatomical landmarks. In such embodiments, the AI architecture and/or ML model can be the same or different than the AI architecture and/or ML model used to perform other operations of the method 400.

The method 400 can continue at block 416 by identifying target points at the target landmarks in each frame. This may include, for example, identifying a center point, an edge, or another suitable portion of the target landmark. As a first example, the target landmarks can include the cul de sac and the anorectal junction, and identifying a target point can include identifying a center point of the cul de sac and a center point of the anorectal junction. As a second example, the target landmarks can include the cul de sac and the levator plate, and identifying a target point can include identifying a center point of the cul de sac and a center point of the levator plate. The foregoing are provided by way of example only—in some embodiments other target anatomical landmarks and/or other target points are used. Similar to the other operations of the method 400, the operation of identifying target points at the target landmarks can be performed using a trained AI architecture and/or ML model, which can be the same or different than the AI architecture and/or ML model used to perform other operations of the method 400. An example of identifying target points at target anatomical landmarks is described below with reference to FIG. 5B.

After the target points are located at block 416, the method 400 can continue at block 418 by measuring a distance between the target points on each frame. For example, in embodiments in which the target points include a center of the cul de sac and a center of the anorectal junction, the operation at block 418 can include measuring a distance between the center of the cul de sac and the center of the anorectal junction. In embodiments in which the target points include a center of the cul de sac and a center of the levator plate, the operation at block 418 can include measuring a distance between the center of the cul de sac and the center of the levator plate. Regardless of the target points, in some embodiments the distance is a horizontal distance, as described below with reference to FIG. 5C. In some embodiments, the distance corresponds to (e.g., is an approximate of) the length of the infra-peritoneal rectum and/or a length of the rectovaginal septum. Similar to the other operations of the method 400, the operation of measuring a distance between the target points can be performed using a trained AI architecture and/or ML model (e.g., find target point(s) using object detection ML model, and measure distance (e.g., in pixels) between target point(s)), which can be the same or different than the AI architecture and/or ML model used to perform other operations of the method 400.

After the distances are measured at block 418, the method 400 can continue at block 420 by determining a compression ratio for the patient. This may include (a) selecting the maximum distance measured in an individual frame at block 418, and (b) selecting the minimum distance measured in an individual frame at block 418. The maximum distance can correspond to the maximum length of the infra-peritoneal rectum and/or rectovaginal septum (e.g., the length of the infra-peritoneal rectum and/or the rectovaginal septum at rest), and the minimum distance can correspond to a minimum length of the infra-peritoneal rectum and/or rectovaginal septum (e.g., the length of the infra-peritoneal rectum and/or rectovaginal septum during Valsalva). The compression ratio can be calculated based on the maximum infra-peritoneal rectum length/rectovaginal septum length and the minimum infra-peritoneal rectum length/rectovaginal septum length using one of equations (1)-(3) described above in Section A. Similar to the other operations of the method 400, the operation of determining a compression ratio for the patient may be performed using a trained AI architecture and/or ML model, which can be the same or different than the AI architecture and/or ML model used to perform other operations of the method 400.

Based on the determined compression ratio at block 420, the method 400 can continue at block 422 by determining whether the patient is a candidate for ODS surgery based, at least in part, on the determined compression ratio. For example, the compression ratio can be compared against a threshold compression ratio. If the compression ratio exceeds the threshold compression ratio, then the patient has a hypermobile rectum and would likely benefit from surgical intervention. If the compression ratio does not exceed the threshold compression ratio, then the patient does not have a hypermobile rectum and is less likely to benefit from surgical intervention.

In some embodiments, the threshold compression ratio can be between about 5 and about 30, or between about 5 about 25, or between about 10 and about 20. Representative threshold compression ratios that can be used include, but are not limited to, 10, 11, 12, 13, 14, 15, 16, 17, 18, 19, or 20. The operation of identifying whether the patient is a candidate for ODS surgery based on the compression ratio may also be performed using a trained AI architecture and/or ML model, which can be the same or different than the AI architecture and/or ML model used to perform other operations of the method 400.

Without being bound by theory, using one or more AI architectures and/or ML models to perform various operations of the method 400 is expected to improve both the accuracy and efficiency of using dynamic ultrasound in the diagnosis of ODS. For example, using one or more AI architectures and/or ML models to provide real-time feedback about probe positioning (e.g., as described at blocks 404 and 408) is expected to permit ultrasound technicians untrained at performing dynamic ultrasound on ODS patients to nevertheless obtain quality imaging data that can be used to determine a treatment plan for the patient. As a second example, using one or more AI architectures and/or ML models to identify target landmarks (block 412), identify target points (block 416), measure distances (418), and calculate compression ratios (420) is expected to increase the speed that patients can be diagnosed with ODS (e.g., instead of waiting for manual review of ultrasound data), improve the accuracy of diagnosis (e.g., avoiding human error), allow for more data to be analyzed, which in turn is expected to lead to more accurate diagnoses, among other improvements and advantages. The systems and methods described herein may also provide additional advantages not expressly described herein.

FIG. 5A illustrates a representative ultrasound frame 500 including representative target anatomical landmarks, in accordance with some embodiments of the present technology. For example, the frame 500 includes a plurality of target anatomical landmarks, including a cul de sac CDS, a levator plate LP, and a rectum R. In other embodiments, other target anatomical landmarks can be identified in the frame 500.

FIG. 5B illustrates the representative ultrasound frame 500 shown in FIG. 5B with target points identified for several of the target anatomical landmarks, in accordance with some embodiments of the present technology. For example, the frame 500 includes two target points: a center point 502 of the cul de sac CDS and a center point 504 of the levator plate LP. In other embodiments, other target points for the target anatomical landmarks can be identified.

FIG. 5C illustrates obtaining a measurement associated with the target points of the target anatomical landmarks, in accordance with some embodiments of the present technology. For example, FIG. 5C illustrates a horizontal distance Z between the center point 502 of the cul de sac CDS and the center point 504 of the levator plate LP. As previously described, the cul de sac CDS and the levator plate LP can be positioned at opposite ends of the infra-peritoneal rectum and the rectovaginal septum, and so the horizontal distance Z can approximate a length of the infra-peritoneal rectum and/or rectovaginal septum. Accordingly, the horizontal distance Z can be used to calculate the compression ratio for the patient, as previously described herein. In other embodiments, other measurements associated with the target points can be obtained, such as a linear distance between the center point 502 of the cul de sac CDS and the center point 504 of the levator plate LP.

Although described in the context of diagnosing and evaluating patients with ODS, the present technology can also be used to improve the efficiency and effectiveness of dynamic ultrasound in the evaluation and diagnosis of other patient conditions and/or anatomical structures. For example, in some embodiments the present technology can be used to diagnose and/or evaluate urinary incontinence using dynamic endovaginal ultrasound. In such embodiments, the target anatomical landmarks may include, but are not limited to, the urethral meatus, pubic symphysis, bladder neck, and/or other pelvic floor structures, and the measurements associated with the target anatomical landmarks may include, but are not limited to, urethral length, retropubic bladder neck angle, urethral anterior-posterior diameter, bladder neck-pubic bone angle, meatus-pubic bone angle, urethral knee-pubic bone angle, and/or urethral knee-pubic bone distance. Similar to the techniques described above with respect to evaluating ODS patients, the foregoing measurements can be automatically calculated and compared under a variety of patient conditions (e.g., at rest, under strain/Valsalva, etc.) using AI architectures and/or ML models to provide objective diagnosis criteria that can subsequently be used to determine appropriate treatments.

### C. Examples

Several aspects of the present technology are set forth in the following examples:

1. A computer-implemented method for providing medical care to a patient, the method comprising:
obtaining a plurality of ultrasound frames of the patient, wherein each frame includes an ultrasound image of a pelvic region of the patient;
using one or more trained AI architectures and/or ML models to:
   identify two or more target anatomical landmarks in at least a subset of the plurality of frames,
   identify a target point at each of the two or more target anatomical landmarks in at least the subset of the plurality of frames,
   for each individual frame of the subset of the plurality of frames, measure a distance between the target points identified in the individual frame, and
   determine a compression ratio for the patient based on the measured distances; and
displaying the determined compression ratio and/or an indication of whether the patient is a candidate for surgery based, at least in part, on the determined compression ratio.

2. The computer-implemented method of example 1 wherein the two or more anatomical landmarks include two or more of a rectum, a rectovaginal septum, a cul de sac, a levator plate, and/or an anorectal junction.

3. The computer-implemented method of example 2 wherein the two or more anatomical landmarks include the cul de sac and the anorectal junction.

4. The computer-implemented method of example 2 wherein the two or more anatomical landmarks include the cul de sac and the levator plate.

5. The computer-implemented method of any of examples 1~4 wherein the target points include respective center points of each of the two or more target anatomical landmarks.

6. The computer-implemented method of any of examples 1-5 wherein determining the compression ratio based on the measured distances includes:
selecting the maximum distance measured in an individual frame;
selecting the minimum distance measured in an individual frame; and
using the maximum distance and the minimum distance to calculate the compression ratio.

7. A computer-implemented method for providing medical care to a patient, the method comprising:
receiving an ultrasound video of a pelvic region of a patient;
using one or more trained AI architectures and/or ML models to automatically analyze the ultrasound video to detect the presence or absence of two or more anatomical landmarks;
if the ultrasound video does not include the two or more anatomical landmarks, providing feedback for modifying a position of an ultrasound probe; and
if the ultrasound video does include the two or more anatomical landmarks, automatically converting the ultrasound video into individual frames.

8. The computer-implemented method of example 7 wherein the two or more anatomical landmarks include two or more of a rectum, a rectovaginal septum, a cul de sac, a levator plate, and/or an anorectal junction.

9. The computer-implemented method of example 7 or 8 wherein the feedback includes instructions to advance the probe deeper into the patient by a specific distance, retract the probe from the patient by a specific distance, rotate the probe a specific number of degrees, adjust an angle of the probe, and/or adjust a setting of a signal emitted from the probe.

10. The computer-implemented method of any of examples 7-9 wherein the operations of receiving, analyzing, and providing feedback are performed in real time or substantially real time.

11. A system for providing medical care to a patient, the system comprising:
a processor; and
a memory storing instructions that, when executed by the processor, cause the system to:
   obtain a plurality of ultrasound frames of the patient, wherein each frame includes an ultrasound image of a pelvic region of the patient;
   use one or more trained AI architectures and/or ML models to:
      identify two or more target anatomical landmarks in at least a subset of the plurality of frames,
      identify a target point at each of the two or more target anatomical landmarks in at least the subset of the plurality of frames,
      for each individual frame of the subset of the plurality of frames, measure a distance between the target points identified in the individual frame, and
      determine a compression ratio for the patient based on the measured distances; and
   displaying the determined compression ratio and/or an indication of whether the patient is a candidate for surgery based, at least in part, on the determined compression ratio.

12. The system of example 11 wherein the two or more anatomical landmarks include two or more of a rectum, a rectovaginal septum, a cul de sac, a levator plate, and/or an anorectal junction.

13. The system of example 12 wherein the two or more anatomical landmarks include the cul de sac and the anorectal junction.

14. The system of example 12 wherein the two or more anatomical landmarks include the cul de sac and the levator plate.

15. The system of any of examples 11-14 wherein the target points include respective center points of each of the two or more target anatomical landmarks.

16. The system of any of examples 11-15 wherein the operation of determining the compression ratio based on the measured distances includes:
selecting the maximum distance measured in an individual frame;
selecting the minimum distance measured in an individual frame; and
using the maximum distance and the minimum distance to calculate the compression ratio.

17. The system of any of examples 11-16, further comprising the one or more AI architectures and/or ML models.

18. The system of any of examples 11-16 wherein the system includes a patient imaging system, and wherein the patient imaging system includes the processor and the memory.

19. The system of any of examples 11-16 wherein the system includes a remote computing system, and wherein the remote computing system includes the processor and the memory.

20. A system for providing medical care to a patient, the system comprising:
a processor; and
a memory storing instructions that, when executed by the processor, cause the system to:
   receive an ultrasound video of a pelvic region of a patient;
   use one or more trained AI architectures and/or ML models to automatically analyze the ultrasound video to detect the presence or absence of two or more anatomical landmarks;
   if the ultrasound video does not include the two or more anatomical landmarks, provide feedback for modifying a position of an ultrasound probe; and
   if the ultrasound video does include the two or more anatomical landmarks, automatically convert the ultrasound video into individual frames.

21. The system of example 20 wherein the two or more anatomical landmarks include two or more of a rectum, a rectovaginal septum, a cul de sac, a levator plate, and/or an anorectal junction.

22. The system of example 20 or 21 wherein the feedback includes instructions to advance the probe deeper into the patient by a specific distance, retract the probe from the patient by a specific distance, rotate the probe a specific number of degrees, adjust an angle of the probe, and/or adjust a setting of a signal emitted from the probe.

23. The system of any of examples 20-22 wherein the operations of receiving, analyzing, and providing feedback are performed in real time or substantially real time.

24. The system of any of examples 20-23, further comprising the one or more AI architectures and/or ML models.

25. The system of any of examples 20-24 wherein the system includes a patient imaging system, and wherein the patient imaging system includes the processor and the memory.

26. The system of any of examples 20-24 wherein the system includes a remote computing system, and wherein the remote computing system includes the processor and the memory.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A computer-implemented method for providing medical care to a patient, the method comprising:
obtaining a plurality of ultrasound frames of the patient, wherein each frame includes an ultrasound image of a pelvic region of the patient;
using one or more trained AI architectures and/or ML models to:
identify two or more target anatomical landmarks in at least a subset of the plurality of frames,
identify a target point at each of the two or more target anatomical landmarks in at least the subset of the plurality of frames,
for each individual frame of the subset of the plurality of frames, measure a distance between the target points identified in the individual frame, and
determine a compression ratio for the patient based on the measured distances; and
displaying the determined compression ratio and/or an indication of whether the patient is a candidate for surgery based, at least in part, on the determined compression ratio.

2. The computer-implemented method of claim 1 wherein the two or more anatomical landmarks include two or more of a rectum, a rectovaginal septum, a cul de sac, a levator plate, and/or an anorectal junction.

3. The computer-implemented method of claim 2 wherein the two or more anatomical landmarks include the cul de sac and the anorectal junction.

4. The computer-implemented method of claim 2 wherein the two or more anatomical landmarks include the cul de sac and the levator plate.

5. The computer-implemented method of claim 1 wherein the target points include respective center points of each of the two or more target anatomical landmarks.

6. The computer-implemented method of claim 1 wherein determining the compression ratio based on the measured distances includes:
selecting the maximum distance measured in an individual frame;
selecting the minimum distance measured in an individual frame; and
using the maximum distance and the minimum distance to calculate the compression ratio.

7. A computer-implemented method for providing medical care to a patient, the method comprising:
receiving an ultrasound video of a pelvic region of a patient;
using one or more trained AI architectures and/or ML models to automatically analyze the ultrasound video to detect the presence or absence of two or more anatomical landmarks;
if the ultrasound video does not include the two or more anatomical landmarks, providing feedback for modifying a position of an ultrasound probe; and
if the ultrasound video does include the two or more anatomical landmarks, automatically converting the ultrasound video into individual frames.

8. The computer-implemented method of claim 7 wherein the two or more anatomical landmarks include two or more of a rectum, a rectovaginal septum, a cul de sac, a levator plate, and/or an anorectal junction.

9. The computer-implemented method of claim 7 wherein the feedback includes instructions to advance the probe deeper into the patient by a specific distance, retract the probe from the patient by a specific distance, rotate the probe a specific number of degrees, adjust an angle of the probe, and/or adjust a setting of a signal emitted from the probe.

10. The computer-implemented method of claim 7 wherein the operations of receiving, analyzing, and providing feedback are performed in real time or substantially real time.

11. A system for providing medical care to a patient, the system comprising:
a processor; and
a memory storing instructions that, when executed by the processor, cause the system to:
obtain a plurality of ultrasound frames of the patient, wherein each frame includes an ultrasound image of a pelvic region of the patient;
use one or more trained AI architectures and/or ML models to:
identify two or more target anatomical landmarks in at least a subset of the plurality of frames,
identify a target point at each of the two or more target anatomical landmarks in at least the subset of the plurality of frames,
for each individual frame of the subset of the plurality of frames, measure a distance between the target points identified in the individual frame, and
determine a compression ratio for the patient based on the measured distances; and
displaying the determined compression ratio and/or an indication of whether the patient is a candidate for surgery based, at least in part, on the determined compression ratio.

12. The system of claim 11 wherein the two or more anatomical landmarks include two or more of a rectum, a rectovaginal septum, a cul de sac, a levator plate, and/or an anorectal junction.

13. The system of claim 12 wherein the two or more anatomical landmarks include the cul de sac and the anorectal junction.

14. The system of claim 12 wherein the two or more anatomical landmarks include the cul de sac and the levator plate.

15. The system of claim 11 wherein the target points include respective center points of each of the two or more target anatomical landmarks.

16. The system of claim 11 wherein the operation of determining the compression ratio based on the measured distances includes:
selecting the maximum distance measured in an individual frame;
selecting the minimum distance measured in an individual frame; and
using the maximum distance and the minimum distance to calculate the compression ratio.

17. The system of claim 11, further comprising the one or more AI architectures and/or ML models.

18. The system of claim 11 wherein the system includes a patient imaging system, and wherein the patient imaging system includes the processor and the memory.

19. The system of claim 11 wherein the system includes a remote computing system, and wherein the remote computing system includes the processor and the memory.

20. A system for providing medical care to a patient, the system comprising:
a processor; and
a memory storing instructions that, when executed by the processor, cause the system to:
receive an ultrasound video of a pelvic region of a patient;
use one or more trained AI architectures and/or ML models to automatically analyze the ultrasound video to detect the presence or absence of two or more anatomical landmarks;
if the ultrasound video does not include the two or more anatomical landmarks, provide feedback for modifying a position of an ultrasound probe; and
if the ultrasound video does include the two or more anatomical landmarks, automatically convert the ultrasound video into individual frames.

21. The system of claim 20 wherein the two or more anatomical landmarks include two or more of a rectum, a rectovaginal septum, a cul de sac, a levator plate, and/or an anorectal junction.

22. The system of claim 20 wherein the feedback includes instructions to advance the probe deeper into the patient by a specific distance, retract the probe from the patient by a specific distance, rotate the probe a specific number of degrees, adjust an angle of the probe, and/or adjust a setting of a signal emitted from the probe.

23. The system of claim 20 wherein the operations of receiving, analyzing, and providing feedback are performed in real time or substantially real time.

24. The system of claim 20, further comprising the one or more AI architectures and/or ML models.

25. The system of claim 20 wherein the system includes a patient imaging system, and wherein the patient imaging system includes the processor and the memory.

26. The system of claim 20 wherein the system includes a remote computing system, and wherein the remote computing system includes the processor and the memory.
